(19) United States
(12) Patent Application Publication
(10) Pub. No.: US 2009/0227546 A1
(43) Pub. Date: Sep. 10, 2009

(54) PHARMACEUTICAL COMPOSITION OF DROSPIRENONE AND ETHYNYLESTRADIOL

(75) Inventors: Carlos Ariel Sandrone, Buenos Aires (AR); Jose Mario Sakson, Buenos Aires (AR); Maria del Carmen Cajarville Basaistegui, Montevideo (UY); Jose Daniel Larrosa Pomi, Canelones (UY)

Correspondence Address: BERENBAUM, WEINSHIENK & EASON, P.C, 370 17TH STREET, SUITE 4800, DENVER, CO, 80202, US

(73) Assignee: Laboratories Liconse, S.A., Barcelona (ES)

(21) Appl. No.: 11/573,499

(22) PCT Filed: Aug. 2, 2005
(86) PCT No.: PCT/EP05/53759

§ 371 Date: Feb. 9, 2007

(30) Foreign Application Priority Data
Aug. 9, 2004 (EP) 04103837.3

(51) Int. Cl. A61K 31/56

(52) U.S. Cl. 514/170

(57) ABSTRACT

A pharmaceutical composition of drospirenone and ethynylestradiol with an improved dissolution rate. A method of preparation of a pharmaceutical formulation of drospirenone and ethynylestradiol in order to improve its dissolution profile. The formulation can be used to produce an anovulatory effect when administered correctly in humans.

The present disclosure refers to a pharmaceutical composition of drospirenone and ethynylestradiol with an improved dissolution rate. In addition, described here is a method of preparation of a pharmaceutical formulation of drospirenone and ethynylestradiol in order to improve its dissolution profile.

The formulation may be used to produce an anovulatory effect when administered correctly in humans.

### BACKGROUND

It is known that the estrogenic and progestagen sexual hormones are practically insoluble in water or are so slowly dissolved that their biopharmaceutical properties are directly affected by this behavior or characteristic.

In particular, the synthetic progestagen drospirenone, or 6β,7β,15β,16β-dimethylene-3-oxo-17α-pregn-4-ene-21,17-carbolactone, known from the patents DE3022337 and DE2652761, has a “spatula” form in its crystalline state as can be seen in the microphotographs in FIG. 1 herein magnified 200× with a size no less than 50 microns. The dissolution rate of this crystal in oral pharmaceutical formulations such as tablets shows a very slow dissolution rate as shown in FIG. 17 herein (see batches LP01, LP02 and LP03).

The published application EP1216712A1 discloses the preparation of inclusion complexes between cyclodextrin and drospirenone with the aim of increasing the solubility of drospirenone in water.

The bioavailability of a drug can be limited by a poor dissolution of the same in corporal aqueous fluids, particularly gastric fluids, immediately after administration. The dissolution rate may therefore be critical to rapidly reach therapeutically effective drug levels or in addition, as is the case for drospirenone, it may be influenced by the degradation of the active ingredient due to prolonged contact with gastric fluids with a very low pH, as described in the application WO01/15701 (in patent family with EP1214076B1).

This behavior is seen in vitro, in an acidic medium (HCl 0.1 N), for samples of drospirenone with different particle sizes (see FIG. 16 herein). It can be observed that the presence of the drug in the acidic medium decreases rapidly, after an initial dissolution, regardless of the size of the particles. Drospirenone's bioavailability is therefore doubly affected by the low dissolution rate in aqueous medium and by rapid degradation due to prolonged contact with gastric fluids with a very low pH.

The published patent EP1214076B1 discloses a pharmaceutical composition containing drospirenone and ethynylestradiol where the drospirenone is in a micronized form so that the particles of the active substance have a superficial area of more than 10.000 cm²/g, and a distribution of the size of the particles determined under a microscope where no more than two particles in a given batch have a diameter of more than 30 μm and preferably 20 particles or less have a diameter between 10 μm and 30 μm, thus increasing its dissolution rate particularly for use in oral contraception.

Further the final part of section [0017] of EP1214076B1 reads “Instead of providing the drospirenone in micronized form, it is possible to dissolve it in a suitable solvent, e.g. methanol or ethyl acetate, and spray it onto the surface of inert carrier particles followed by incorporation of the particles containing drospirenone on their surface in the composition.”

However, this section stands alone in the description of EP1214076B1. All working examples plus independent main claims refers to the micronized formulation.

### SUMMARY

A problem which may be solved by the composition and method is to provide a pharmaceutical composition of drospirenone and ethynylestradiol with an improved dissolution rate in water and bodily fluids and improved bioavailability.

As described herein, with a pharmaceutical composition of drospirenone and ethynylestradiol in an amorphous form, without the presence of a crystalline state, the dissolution profiles of both active ingredients may improve, thus increasing their bioavailability.

Further, as described herein, use of the volatile solvent methylene chloride and in particular use of the combination of a mixture of the solvents methylene chloride and methanol may give significant improved dissolution rate results over the use of methanol alone. For further details see e.g. Table 2 of Example 4 and Table 3 of Example 5 herein. Use of methanol alone is theoretically suggested in the prior art document EP1214076B1 (see above).

As explained herein, the volatile solvent methylene chloride may be used to dissolve the drugs. In the subsequent granulation step, the mixture is dried until the residual level of solvent is very low (see Example 1). However, even though such levels may be low, it may still be possible to measure small amounts of the volatile solvent of interest in the final pharmaceutical composition. This may be done by use of gas chromatography (GC) and mass spectrometry (MS).

Therefore, a first aspect hereof may refer to a pharmaceutical composition of drospirenone and ethynylestradiol in an amorphous form, adsorbed on a therapeutically inert solid support, in combination with pharmaceutically acceptable excipients, where the composition contains measurable amounts of the volatile solvent methylene chloride.

It may be preferable to use a mixture of the solvents methylene chloride and methanol. Accordingly, the composition of the first aspect may be a composition with measurable amounts of both of the volatile solvents methylene chloride and methanol.

The dissolution rate of drospirenone and ethynylestradiol may be increased by introducing changes in their crystalline structure, so that the energy needed to dissolve the drug may be much lower. In this case, the dissolution rate may increase considerably, thus achieving an increase in the drugs absorption rate. This way, the time the drugs are in contact with the gastric medium where the pH is too low may be reduced, thus avoiding the eventual degradation reactions of the sensitive drugs by hydrolysis, as described above.

It is therefore an aim to provide a pharmaceutical composition that contains drospirenone and ethynylestradiol in an amorphous form, substantially without the presence of the crystalline state, preferably beginning with a non-micronized drug and using adequate dissolution/granulation solvents in its preparation.

Surprisingly, it has been discovered that the greatest dissolution rate may be obtained by solubilization of drospirenone and ethynylestradiol in the appropriate combination of a mixture of the solvents methylene chloride and methanol and subsequent adsorption on adequate pharmaceutically inert excipients.

It has been observed that the use of another solvent, such as ethanol, even with heat, despite dissolving the drug, may not allow the application on pharmaceutically adequate excipients.

Further, use of methylene chloride and in particular use of the combination of a mixture of the solvents methylene chloride and methanol may give improved results over the use of methanol alone.

The new pharmaceutical composition described herein may provide a rapid dissolution of drospirenone and ethynylestradiol in water as the reference solvent.

More particularly, a solid particulate composition of drospirenone and ethynylestradiol in an amorphous state may be adsorbed on a therapeutically inert support, with some components being soluble in water and others being insoluble in water, characteristics which facilitate a rapid disintegration of the pharmaceutical formulation.

Even more particularly an additional aim of the pharmaceutical composition may be improved dissolution of drospirenone and ethynylestradiol, wherein a solid particulate formulation of an adsorbate of drospirenone and ethynylestradiol in an amorphous state may be adsorbed on a solid, therapeutically inert, support, in combination with pharmaceutically acceptable excipients.

The solid particulate composition may consist of particles selected from the following group of materials or adequate mixtures thereof: corn starch, pregelatinized starch, lactose, Croscarmellose sodic, yellow iron oxide and povidone covered with drospirenone and ethynylestradiol in an amorphous state.

Furthermore, another aim described herein is the preparation process of a pharmaceutical composition of drospirenone and ethynylestradiol, where the active compounds may be found in an amorphous state and substantially free of crystalline shapes.

Consequently, a method for the preparation of a new pharmaceutical composition of drospirenone and ethynylestradiol, may utilize the following steps: i) dissolving drospirenone and ethynylestradiol in a volatile solvent or mixture of volatile solvents, ii) optionally adding a water-soluble polymer, iii) mixing until dissolution, iv) applying the resulting solution onto the solid, therapeutically inert support of particles, and v) drying the resulting adsorbate. One of the volatile solvent(s) of step (i) may be methylene chloride. A water-soluble polymer may be added in step (ii).

The active compounds drospirenone and ethynylestradiol, used in the preparation of a pharmaceutical composition hereof may be in the form of non-micronized particles, with an average diameter no less than about 50 μm and a specific surface area of less than approximately 5000 cm²/g measured by the BET technique.

Advantageously, the use of non-micronized forms may reduce the environmental inconveniences associated with an additional stage in the manipulation of hormones, such as the process of micronization.

A third aspect relates to a pharmaceutical preparation of a number of dose units for daily oral administration for a period of at least about 21 consecutive days, where said dose units may be from about 1 to about 4 mg of drospirenone and from about 0.01 to about 0.05 mg of ethynylestradiol, wherein that said drospirenone and ethynylestradiol may be in an amorphous form, adsorbed on a therapeutically inert solid support, of the first aspect hereof or implementations thereof as described herein, in combination with pharmaceutically acceptable excipients.

Finally, described herein is the use of drospirenone combined with ethynylestradiol for preparing a pharmaceutical composition, for the anovulation of a mammal, in particular a human, the composition including an amount of drospirenone corresponding to a daily dosage, on administration of the composition, of from about 1 mg to about 4 mg, and comprising an amount of ethynylestradiol corresponding to a daily dosage, on administration of the composition, of from about 0.01 to about 0.05 mg.

This use may alternatively be formulated as a method for the anovulation of a mammal, in particular a human, that may include administering a pharmaceutical composition, made up of a number of dose units for daily oral administration, where said dose units consist of approximately 1 mg to approximately 4 mg drospirenone and approximately 0.01 to approximately 0.05 mg of ethynylestradiol, in which the drospirenone and ethynylestradiol may be in an amorphous shape, adsorbed onto a solid therapeutically inert support, in combination with acceptable pharmaceutical excipients.

### BRIEF DESCRIPTION OF THE FIGURES

FIG. 1 shows microphotographs of drospirenone in its crystalline state (magnified 200×);

FIG. 2A shows a microphotograph of a lactose particle;

FIG. 2B shows a microphotograph of a group of lactose particles;

FIG. 2C shows a microphotograph of a particle of corn starch;

FIG. 2D shows a microphotograph of a group of corn starch particles;

FIG. 2E shows a microphotograph of a particle of pregelatinized starch;

FIG. 2F shows a microphotograph of a group of pregelatinized starch particles;

FIG. 2G shows a microphotograph of a particle of sodic Croscarmellose;

FIG. 2H shows a microphotograph of a group of sodic Croscarmellose particles;

FIG. 2I shows a microphotograph of a particle of yellow iron oxide;

FIG. 2J shows a microphotograph of a group of yellow iron oxide particles;

FIG. 2K shows a microphotograph of a particle of Povidone;

FIG. 2L shows a microphotograph of a group of Povidone particles;

FIG. 3 shows a microphotograph of a physical mixture of the two distinct components of the adsorbate;

FIGS. 4A and 4B show microphotographs of the adsorbate of drospirenone and ethynylestradiol on the components that are adequate for obtaining the tablets;

FIG. 5 shows a graph of the DSC of lactose alone;

FIG. 6 shows a graph of the DSC of drospirenone;

FIG. 7 shows a graph of the DSC of magnesium stearate;

FIG. 8 shows a graph of the DSC of corn starch;

FIG. 9 shows a graph of the DSC of pregelatinized starch;

FIG. 10 shows a graph of the DSC of yellow iron oxide;

FIG. 11 shows a graph of the DSC of sodic Croscarmellose (Ac-Di-Sol);

FIG. 12 shows a graph of the DSC of Povidone;

FIG. 13 shows a graph of the DSC of placebo (mixture of the excipient components with the absence of ethynylestradiol and drospirenone);

FIG. 14 shows a graph of the DSC of a simple mixture of the components of the adsorbate;

FIG. 15 shows a graph of the DSC of the granules of Example 1 which contains drospirenone and ethynylestradiol adsorbed on the inert excipients;

FIG. 16 shows the dissolution profiles for tablets of drospirenone in a strongly acidic aqueous medium HCl 01 N;

FIG. 17 shows the dissolution profiles for tablets of a composition hereof containing amorphous drospirenone adsorbed on inert excipients, compared to those of compositions containing crystalline drospirenone of various-sized particles; and,

FIG. 18 shows the dissolution profiles for tablets of a composition hereof containing amorphous ethynylestradiol adsorbed on inert excipients, compared to those of compositions containing crystalline ethynylestradiol of various-sized particles.

### DETAILED DESCRIPTION

### A Pharmaceutical Composition of Drospirenone and Ethynylestradiol and a Method for Preparing it

In agreement with the first aspect, the present composition may be a solid particulate formulation of drospirenone and ethynylestradiol in an amorphous state adsorbed on a pharmaceutically acceptable and therapeutically inert support.

The term “adsorbed on a therapeutically inert solid support” may herein also be termed “adsorbed on a therapeutically inert solid base” and the terms may herein be used interchangeably.

The solid particulate formulation may contain particles of pharmaceutically acceptable excipients covered with drospirenone and ethynylestradiol in an amorphous state and ready to be compressed into their definitive pharmaceutical formulation.

The composition hereof may be obtained according to the following steps: dissolving drospirenone and ethynylestradiol in a mixture of volatile solvents, ii) adding a polymer that is water-soluble and/or soluble in the volatile solvent or mixture of volatile solvents, iii) mixing until dissolution, iv) applying the resulting solution onto a support of pharmaceutically acceptable and therapeutically inert solid particles, v) drying the resulting adsorbate.

The volatile solvent may be methanol. Preferably, the volatile solvent may be methylene chloride and even more preferable may be a mixture of methylene chloride and methanol, which is acceptable to use in drug manufacturing processes.

Preferably the volatile solvent is about a [9 to 4]:[3 to 0.5] v/v mixture of methylene chloride/methanol. Within the range of mixtures it is preferred that the volatile solvent is about a 6:1 v/v mixture of methylene chloride/methanol or about a 5:2 v/v mixture of methylene chloride/methanol.

The drospirenone may be prepared as described, in for example, U.S. Pat. No. 4,129,564. The drug drospirenone used in the preparation of the pharmaceutical composition hereof may preferably be in the form of non-micronized particles with an average diameter of no less than about 50 μm and a specific surface area of less than approximately 5000 cm²/g obtained by the BET technique.

The estrogen ethynylestradiol may be prepared according to the methods described in GB 516,444, U.S. Pat. No. 2,243,887, U.S. Pat. No. 2,251,939, U.S. Pat. No. 2,265,976 and U.S. Pat. No. 2,267,257. The drug ethynylestradiol used in the preparation of the pharmaceutical composition hereof may preferably be in the form of non-micronized particles with an average diameter no less than about 50 μm and a specific superficial area obtained by the BET Technique of less than approximately 5000 cm²/g.

Water-soluble polymers that may be acceptable for the preparation of the binder solution may be preferably chosen from the polyvinylpyrrolidones as preferred in the method of the invention, more preferably polyvinylpyrrolidone Povidone K 30 (such as that provided by BASF or ISP).

Examples of other suitable water-soluble polymers may include a polymer selected from the group consisting of: Alginic acid, Carbomer, Carboxymethylcellulose Sodium, Carrageenan, Dextrin, Hydroxypropyl Methylcelullose, Polyethylene Glycol, Polymethacrylates, Polyvinyl Alcohol and Povidone K90.

The resulting solution may be applied onto a base of pharmaceutically acceptable and therapeutically inert solid particles. The application may be carried out using a mixer/granulator like a Pony mixer. However, it may also be done by other techniques such as fluid bed based spraying.

The adequate particle materials used to make the base hereof may be chosen from either starch, pregelatinized starch, lactose, sodic Croscarmellose, or yellow iron oxide and mixtures of the same.

The lactose used may be preferably monohydrate lactose, which is commonly used as an excipient and diluent in the pharmaceutical industry. This lactose can be provided by manufacturers such as MEGGLE, QUEST International, or BORCULO Whey Products.

The starch may be from corn or potatoes, and is commonly used as a diluent in the pharmaceutical industry. The starch can be provided by: ORCON, OAVEBE, CERESTAR INT. SALES, GRAIN PROCESSING CORP., PARTICLE DYNAMICs INC., GLUTAL, etc.

The pregelatinized starch may be from corn or potatoes, and is commonly used as a diluent and agglutinant in the pharmaceutical industry. The pregelatinized starch can be provided by: COLORCON, OAVEBE, CERESTAR INT. SALES, GRAIN PROCESSING CORP., PARTICLE DYNAMICs INC., GLUTAL, etc.

The sodic croscarmellose, commonly used as a superdisintegrant in the pharmaceutical industry, may be provided by COLORCON, ALLCHEM INTERNATIONAL, METSA SPECIALITY CHEMICAL LTD, AVEBE, etc.

The yellow iron oxide, commonly used as a coloring material in the pharmaceutical industry, can be provided by COLORCON.

The magnesium stearate, commonly used as a lubricant in the pharmaceutical industry, may be provided by LE STAR QUIMICA.

The granules obtained may be dried using any known drying method within the art, such as drying in a static bed dryer.

The drying may be carried out until obtaining a granule with residual levels of solvent that are about 100 times below that permitted by the International Harmonization Conference on technical requirements for the registration of pharmaceutical products for human use, in its guide Q3C.

By subjecting the granule to drying, the presence of polyvinylpyrrolidone in the drospirenone and ethynylestradiol solution may act by the formation of a tridimensional polymer structure, preventing the crystalline growth of drospirenone and ethynylestradiol, in such a way that the drugs may be deposited onto the excipient particles in an amorphous state. Subsequently, this mixture should only be lubricated in order to compress it and obtain a pharmaceutical formulation that gives the dose unit.

A separate aspect relates to a pharmaceutical composition of drospirenone and ethynylestradiol in amorphous form, adsorbed on a therapeutically inert solid support, in combination with pharmaceutically acceptable excipients obtained by a method for the preparation of a pharmaceutical composition of the second aspect hereof and implementations thereof as described herein.

Particular examples of preparation are given below. The purposes of these are merely illustrative and are not intended to limit the scope of the invention, which is determined by the claims.

### Pharmaceutical Preparation:

The pharmaceutical preparation of the third aspect of the invention may include drospirenone and ethynylestradiol in amorphous form, adsorbed on a solid basis chosen from among particles selected from corn starch, pregelatinized starch, lactose, sodic croscarmellose, yellow iron oxide, polyvinylpyrrolidone or mixtures of one or more of the same, in combination with pharmaceutically acceptable excipients.

In this pharmaceutical preparation, at least about 80% of drospirenone dissolves from said dose units within about 20 minutes, in a test carried out in a dissolution apparatus according to the U.S. Pharmacopoeia, Edition number 27, Apparatus 2, in 900 ml of distilled water at 37° C. and stirred at 50 rpm.

In another pharmaceutical preparation, at least about 80% of ethynylestradiol dissolves from said dose units within about 20 minutes, in a test carried out in a dissolution apparatus according to the U.S. Pharmacopoeia, Edition number 27, Apparatus 2, in 900 ml of distilled water at 37° C. and stirred at 50 rpm.

Compositions particularly adequate for oral administration may include unitary dose formulations such as tablets, capsules, pills, powder in packets, etc. These dose units may contain an amount of drospirenone in the range from approximately 1 mg to approximately 4 mg, with about 3 mg being preferable, and an amount of ethynylestradiol in the range from approximately 0.01 to approximately 0.05 mg, with about 0.02 to about 0.03 mg being preferable.

### A Method for the Anovulation a Mammal:

The composition may alternatively be formulated as a method for the anovulation of a mammal, in particular a human, wherein a pharmaceutical composition is administered in a number of dose units for daily oral administration, where said dose units include approximately 1 mg to approximately 4 mg drospirenone and approximately 0.01 to approximately 0.05 mg of ethynylestradiol, in which the drospirenone and ethynylestradiol may be in an amorphous shape, adsorbed onto a solid therapeutically inert support, in combination with acceptable pharmaceutical excipients.

With respect to the method for the anovulation of a mammal, in particular a human, the daily oral administration may preferably be for a period of at least about 21 consecutive days.

Further, there may be about 7 or fewer said daily dosage units containing no active agent. Alternatively, it is possible to include, in the dosage regimen, a period of about 7 days or less during which no dosage units are ingested.

The number of daily dosage units comprising the combination of drospirenone and ethynylestradiol may be about 21, 22, 23 or 24, and the number of daily dosage units containing no active agent may then be about 7, 6, 5 or 4, as the case may be.

Alternatively, a pharmaceutical composition, as described herein, may be used for hormone replacement therapy (HRT). The one skilled in the art would typically know how to optimise doses and administration with respect to hormone replacement therapy.

### Pharmaceutically Acceptable Excipient

By “pharmaceutically acceptable excipient” it should be understood as any pharmaceutically acceptable conventional vehicle, including but not limited to, binders, tablet desintegrants, diluents, lubricants, tablet glidants, opacifiers of tablets and capsules, colorings, sweeteners, flavoring, antioxidants, buffer agents, among others.

As used here, the expression “binders” may refer to substances used to provoke the adhesion of the dust particles in the granules of the tablets, and include as examples and without being limiting, sodic carboxymethylcellulose, polyvinylpyrrolidone, sugars, ethylcellulose, gelatin, guar gum, polyethylene glycol, polyethylene oxide or combinations of the same and similar ones known to those with common knowledge of the art.

As used here, the expression “tablet desintegrants” may refer to a compound used in solid dosing formulations to promote the rupture of the solid mass into small particles. Examples of desintegrants include, without being limiting, starches, pregelatinized starches, bentonite, microcrystalline cellulose and its esters and salts, calcium or sodium carboxymethylcellulose and its modifications, alginates, reticulated povidone, gums such as guar, agar, pectin and others as are known to those with common knowledge of the art.

As used here, the expression “tablet diluents or filler” may refer to inert substances used as filling materials to create the desired mass, in the preparation of tablets and capsules, and include, as examples and without being limiting, dibasic calcium phosphate, kaolin, lactose, saccharose, mannitol, microcrystalline cellulose, sorbitol, starch and sugars (mono and polysaccharides) and others as known to those with common knowledge in the art.

As used here, the expression “lubricant” may refer to substances used in the formulations of tablets to reduce friction during the compression of the tablets, that include, as examples and without being limiting, calcium stearate, magnesium stearate, mineral oil, stearic acid, 1-Leucine, zinc stearate, talc and others as are known by those with common knowledge in the art.

As used here, the expression “tablet glidants” or “flow promoters” may refer to agents used in the formulations of capsules and tablets to promote the fluidity of the granulation, that include, as examples and without being limiting, colloidal silica, corn starch, talc calcium silicate, magnesium silicate, and others as are known by those with common knowledge in the art.

As is used here, the expression “opacifiers of tablets and capsules” may refer to a compound used to coat a tablet or capsule with an opaque covering combined or not with a coloring, and include, as examples and without being limiting, titanium dioxide, magnesium carbonate, calcium carbonate and others as are known by those with common knowledge of the art.

As is used here, the expression “coloring” may refer to a compound used to give color to the pharmaceutical compositions, as for example and without being limiting, caramel, ferric oxide, beta carotene, carmine, curcumin, coloring bases and their sprays and other materials known by those with common knowledge in the art.

As used here, the expression “sweetening agent” may refer to a compound used to give a preparation a sweet flavor, and include as examples without being limiting, saccharose, dextrose, aspartame, mannitol, sodium saccharin, sorbitol and others as known by those with common knowledge of the art.

As is used here, the expression “flavoring” may refer to a compound used to give flavor and/or a pleasant aroma to a pharmaceutical preparation. Examples of flavoring agents include, without being limiting, flavoring and aromatic oils, synthetic flavorings and/or natural oils, and plant extracts, leaves, flowers, fruits and others or other combinations of the same.

As is used here, the expression “antioxidant” may refer to an agent that prevents the oxidation and therefore the deterioration of the active agent and other components by oxidative processes, and include as examples without being limiting ascorbic acid, ascorbyl palmitate, sodium bisulfate and others as known by those with common knowledge of the art.

As used here, the expression “buffer agent” may refer to a compound used to resist the change in pH when there is dissolving or the addition of acids or alkalines in the composition, and include as examples without being limiting, potassium metaphosphate, potassium phosphate, monobasic sodium acetate, sodium citrate and others as known by those with common knowledge of the art.

It should be understood that the compounds used in the art of pharmaceutical formulations generally serve a variety of functions and objectives, meaning that the agents mentioned are therefore not for a specific function or limited to that function.

### Separate Independent Aspects

As explained herein and demonstrated in working examples herein (see e.g. Table 2 of Example 4 and Table 3 of Example 5) a pharmaceutical composition as described herein may have a very good dissolution profile. More specifically, at least about 80% of drospirenone and ethynylestradiol may dissolve from said composition within about 20 minutes in a test carried out in a recognized standard dissolution apparatus.

This is the first time that a pharmaceutical composition has been described with such an optimized dissolution profile and it is a significant contribution to the art. As explained herein, such a pharmaceutical composition may be obtained by use of methylene chloride as one of the volatile solvents. However a skilled person could further investigate the issue and probably identify other solvents than methylene chloride that could be used to make adequate pharmaceutical compositions and yet remain within the scope hereof.

Accordingly, a separate independent aspect hereof relates to a pharmaceutical composition of drospirenone and ethynylestradiol in amorphous form, adsorbed on a therapeutically inert solid support, in combination with pharmaceutically acceptable excipients, wherein one or both of at least about 80% of drospirenone and at least about 80% of the ethynylestradiol dissolve from said composition within about 20 minutes in a test carried out in a dissolution apparatus according to the U.S. Pharmacopoeia, Edition number 27, Apparatus 2, in 900 ml of distilled water at 37° C. and stirred at 50 rpm.

As discussed above, the pharmaceutical composition of this separate independent aspect of the invention may be made by use of methylene chloride as volatile solvent. However, it may also be made by use of other volatile solvent(s) not involving methylene chloride.

In light of this, another separate independent aspect hereof relates to a method for the preparation of a pharmaceutical composition, of the following steps:
i) dissolving drospirenone and ethynylestradiol in a volatile solvent or mixture of volatile solvents,
ii) optionally, adding a water-soluble polymer,
iii) mixing until dissolution,
iv) applying the resulting solution onto a base of solid particles that are therapeutically inert,
v) drying the obtained granulated adsorbate.

In step ii) a water-soluble polymer may preferably be added. The working examples herein provide guidance in order to identify other (not methylene chloride) adequate volatile solvents. For instance, in comparative example 2B is shown that about 16 ml of methanol alone is not enough to completely dissolve about 3.06 g of drospirenone. This may be used as basis for identifying suitable solvents. One may repeat the dissolution protocol, as described in working example 1 herein, but using a panel of other suitable solvent candidates. The ones that are capable of completely dissolving drospirenone may be preferred solvents.

The volatile solvent or mixture of volatile solvents of step i) may thus preferably be solvent(s) that may be capable of completely dissolving drospirenone in the following dissolution assay:

About 16.0 ml of the volatile solvent or mixture of volatile solvents are placed in a stainless steel stirring apparatus, next, about 0.0306 g of ethynylestradiol (non-micronized with a particle size no less than about 50 μm) are added under constant stirring, the mixture is shaken/stirred until completely dissolved, next about 3.06 g of drospirenone (non-micronized with a particle size no less than about 50 μm) are added under constant stirring until the drospirenone is completely dissolved.

All embodiments described above in relation to the aspects hereof are also preferred implementations in relation to the separate independent aspects of the invention as described in this section.

### EXAMPLES

### Examples for Reference

### Dissolution Profiles of Crystalline Drospirenone in Acidic Medium

The dissolution profiles were obtained from tablets containing drospirenone in a crystalline state with different sized particles, using a dissolution apparatus designated in the U.S. Pharmacopoeia, Edition 27, apparatus 2, at 50 rpm and 900 ml of lauryl sulfate 0.07% in a strongly acidic medium, HCl 0.1 N, thermostatized at 37° C. The results of the amounts of dissolved drug at different time-points are summarized in the following table:

TABLE 1
% of drospirenone dissolved

| | Time (minutes) | | | | | |
|---|---|---|---|---|---|---|
| | 0 | 10 | 20 | 30 | 45 | 60 |
| LP01 | 0 | 50.1% | 47.9% | 37.6% | 33.8% | 26.7% |
| LP02 | 0 | 38.1% | 33.5% | 29.7% | 23.8% | 20.5% |
| LP04 | 0 | 85.3% | 76.5% | 64.6% | 51.1% | 42.2% |

Samples:

LP01: Batch of tablets with non-micronized, crystalline drospirenone with an average particle size of about >100 μm
LP02: Batch of tablets with non-micronized, crystalline drospirenone with an average particle size=about 100 μm
LP04: Batch of tablets with micronized, crystalline drospirenone with an average particle size=about 10 μm

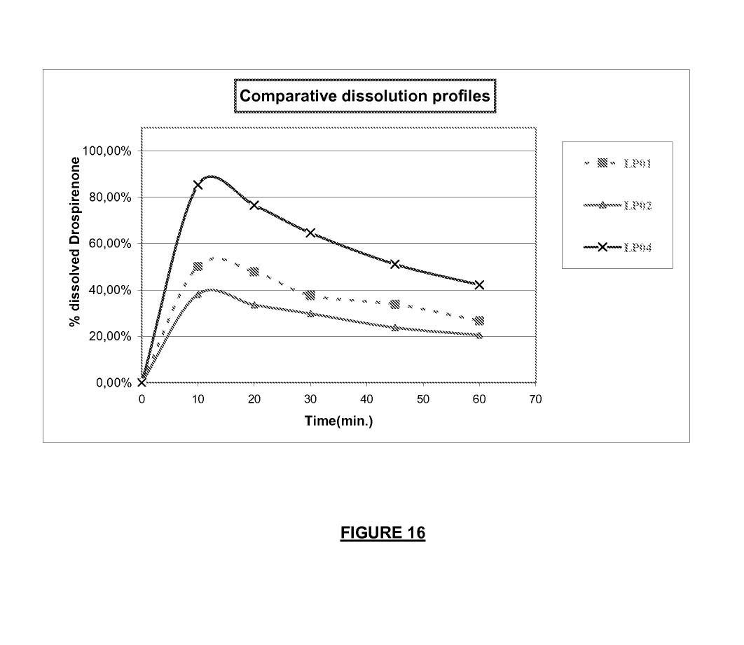

For a better interpretation of the results shown in Table 1, the results are also shown in FIG. 16. From this figure one can see the instability of the drospirenone in an acidic medium, regardless of the size of the particle.

### Example 1

Amount required for producing about 1000 tablets:

Granule:

| | | |
|---|---|---|
| Drospirenone (+ about 2% excess for losses | about 3.0600 | g |
| during the process) | | |
| Ethynylestradiol(+ about 2% excess for losses | about 0.0306 | g |
| during the process) | | |
| Corn starch | about 12.7800 | g |
| Pregelatinized starch | about 15.4400 | g |
| Monohydrate lactose | about 44.0000 | g |
| Sodic croscarmellose | about 0.4000 | g |
| Yellow iron oxide | about 0.0900 | g |
| Povidone | about 3.4000 | g |
| Methylene chloride/methanol (6:1) | about 16.0000 | ml |

Lubrication and final mixture:

| | | |
|---|---|---|
| Sodic Croscarmellose | about 0.4000 | g |
| Magnesium stearate | about 0.4000 | g |

Covering/coating:

| | | |
|---|---|---|
| Aqueous clear lake 5% | about 0.0060 | g |
| (OPADRY YS-1-7006 at 5%) | | |

### II. Preparation Technique

### II.1. Dissolution of the Active Ingredients and Binder

About 16.00 ml of the dichloromethane/methanol solution at about a 6:1 ratio were placed in a stainless steel stirring apparatus. Next, about 0.0306 g of ethynylestradiol, non-micronized with a particle size no less than about 50 μm (addition of an excess of about 2% was foreseen to make up for losses during the process), were added under constant stirring. The mixture was shaken/stirred until completely dissolved. About 3.0600 g of drospirenone, non-micronized with a particle size no less than about 50 μm (addition of an excess of about 2% was foreseen to make up for losses during the process), were added under constant stirring until completely dissolved. Once dissolved, about 3.4000 g of polyvinylpyrrolidone K-30 were added with stirring until completely dissolved.

### II.2. Granulation—Drying

In a planetary-type mixer, the following, which were previously adequately sized by an about 30 mesh net, were added: about 44.0000 g of monohydrate lactose, about 12.7800 g of corn starch, about 15.4400 g of pregelatinized starch, about 0.4000 g of sodic croscarmellose and 0.0900 g of yellow iron oxide. These were mixed for about 15 to about 20 minutes. Next, the previously prepared binder solution that contains the active ingredients was added, and they were kneaded until reaching an adequate consistency for granulating. If necessary, it is possible to add additional solvent (mixture about 6:1 of dichloromethane/methanol). The wet mass was granulated by making it pass through an about 10 mesh net. It was dried in a heater at about 40° C. for about 24 hours, until reaching residual levels of solvent that were about 100 times less than that allowed by the International Conference of Harmonization of technical requirements for the registration of pharmaceutical products for human use, its guide Q3C.

In a separate experiment, the binder solution was applied onto the base of solid particles via spraying. It was done in the following way. In a fluid bed dryer/granulator, previously sized by an about 30 mesh, were added: about 44.0 g of monohydrate lactose, about 12.780 g of corn starch, about 15.44 g of pregelatinized starch, about 0.400 g of sodic croscarmellose and about 0.0900 g of yellow iron oxide. These were mixed for about 15 to about 20 minutes. Then, the previously prepared binder solution that contains the active ingredients was sprayed. The wet mass was dried at about 40° C.

### II.3. Final Mixture—Compression—Covering

The dried granulate was passed through net 18, followed by the addition of about 0.4000 g of sodic croscarmellose and about 0.4000 g of magnesium stearate. These were mixed for about 15 minutes. The resulting granules were compressed into tablets using a rotating machine equipped with a set of flat biconcave punchers of about 13/64″ in diameter at a weight of about 80 mg/tablet and a force of about 6-12 Stron Cobb units.

The compressed tablets were coated with an aqueous clear lake 5%. Name of the batch granulated in the planetary-type mixer: LPDIVuru and name of the batch granulated in the fluid bed dryer: LPDISpray.

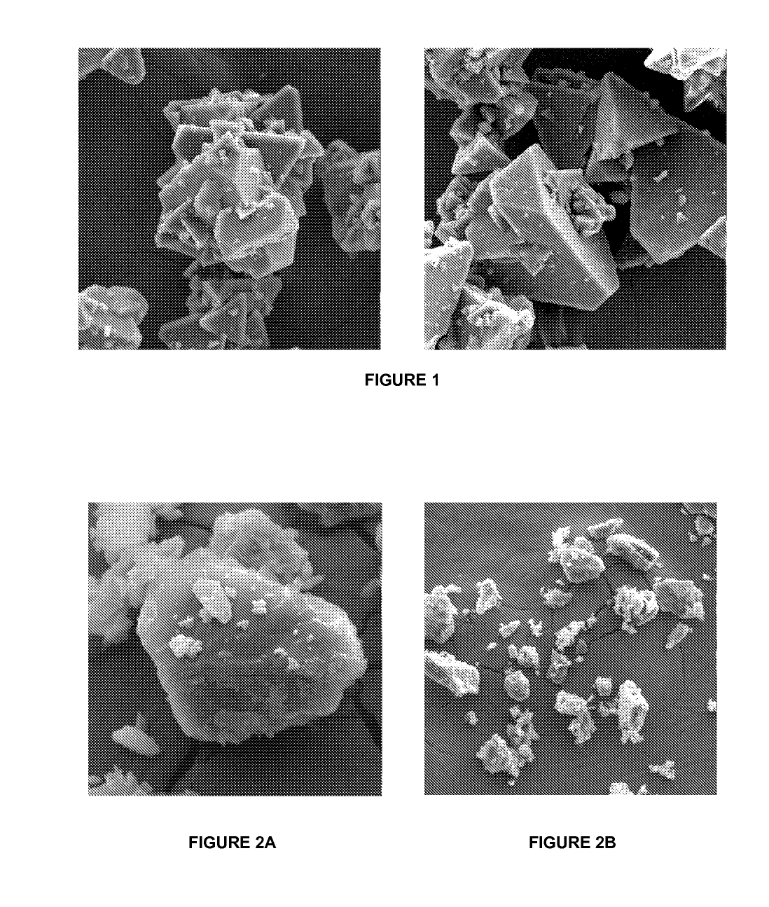

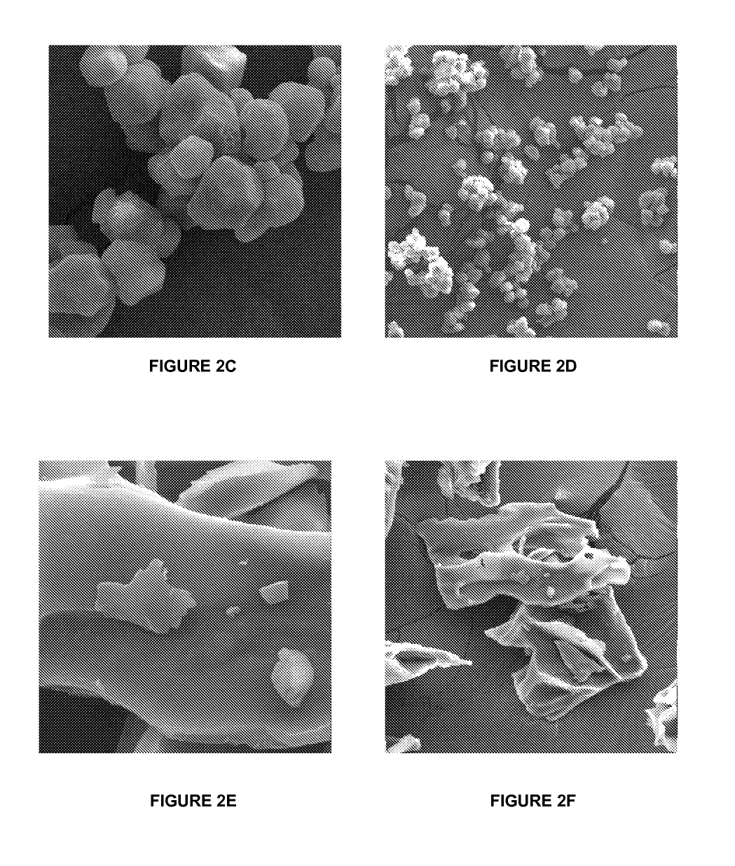

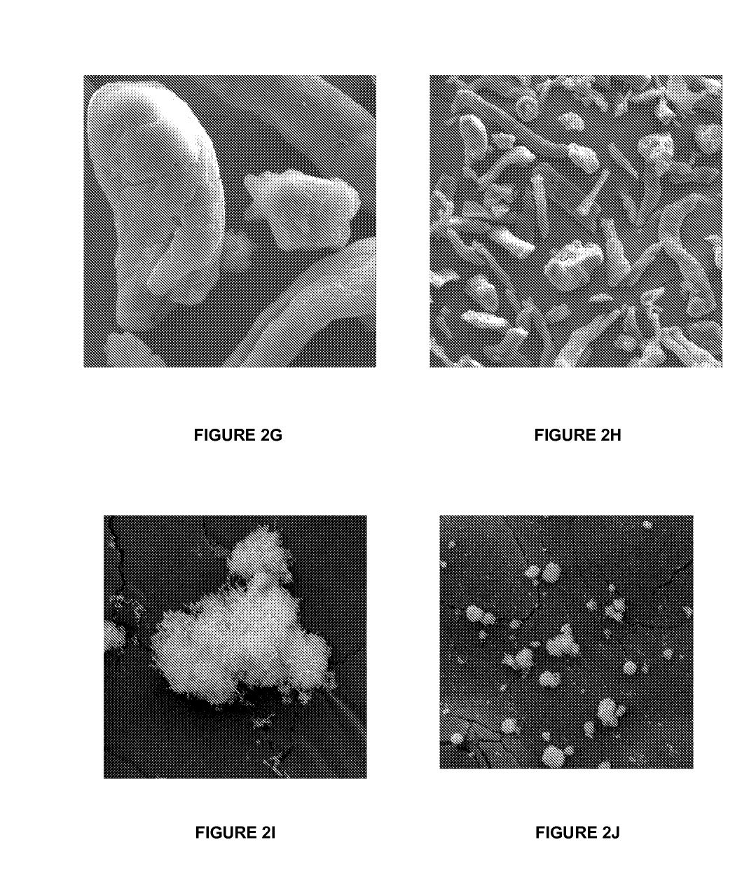

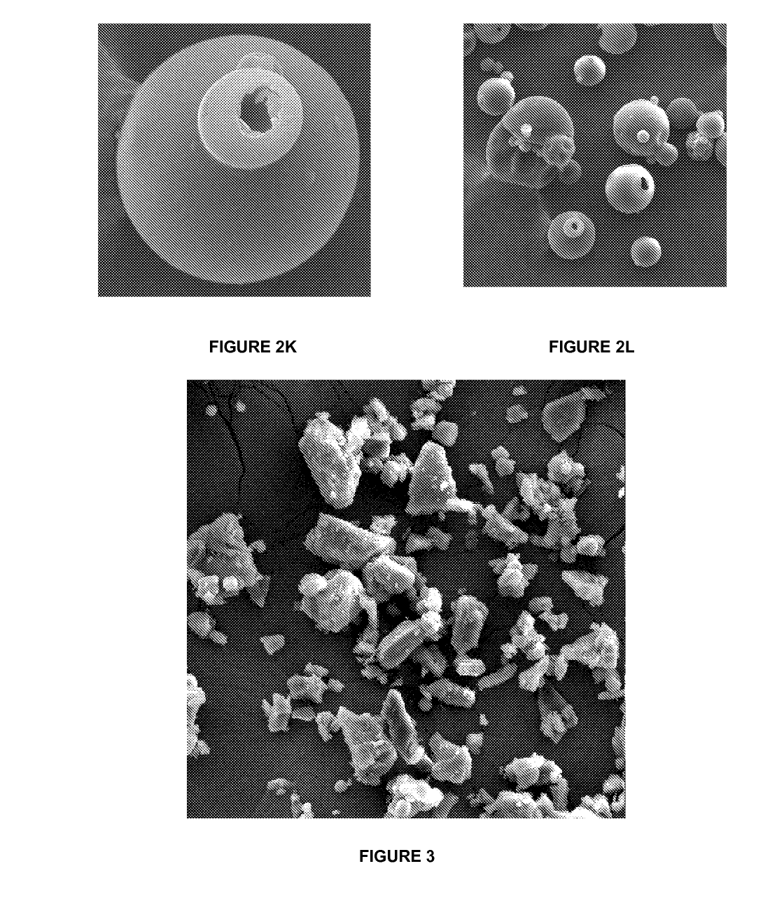

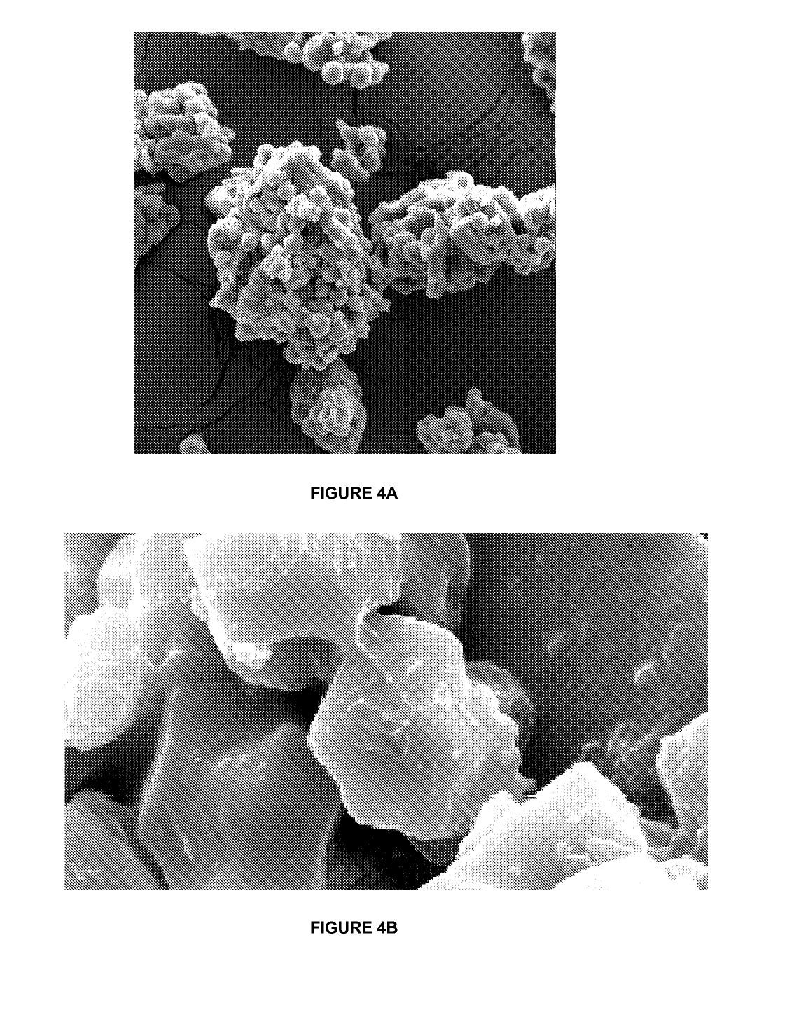

The morphology of the components in each step of the process was analyzed under an electronic microscope. The microphotographs in FIG. 1, magnified 200×, show crystals of drospirenone, with its characteristic “spatula” shape at sizes greater than about 50 μm. FIGS. 2A to 2L show microphotographs of the therapeutically inert materials used as a base for the active agents. In particular, FIGS. 2A and 2B show microphotographs of a lactose particle and of a group of lactose particles, respectively; FIGS. 2C and 2D show microphotographs of a corn starch particle and a group of corn starch particles, respectively; FIGS. 2E and 2F show microphotographs of a particle of pregelatinized starch and a group of pregelatinized starch particles, respectively; FIGS. 2G and 2H show microphotographs of a particle of sodic croscarmellose and a group of sodic croscarmellose particles, respectively; FIGS. 21 and 2J show microphotographs of a particle of yellow iron oxide and a group of yellow iron oxide particles, respectively; FIGS. 2K and 2L show microphotographs of a particle of Povidone and of a group of Povidone particles, respectively. FIG. 3 is a microphotograph of a physical mixture of the different components. FIGS. 4A and 4B are microphotographs of particles of the adsorbate of drospirenone and ethynylestradiol on the inert components, in two views.

### Example 2

A batch of about 1000 tablets were prepared using a process similar to that described in Example 1, where the mixture of volatile solvents methylene chloride/methanol were applied at a proportion of about 5:2. Results similar to those described in Example 1 were obtained.

### Example 2B

This is a comparative example, where only methanol was used as solvent. Only the differences compared to example 1 are given below; the rest was substantially identical to example 1.

Only about 16 ml methanol was used in this example, which compares to substantially the same volume as the about 16 ml methylene chloride/methanol (about 6:1) used in example 1. However, it was found that this about 16 ml quantity of methanol was not enough for the total dissolution of drospirenone because the methanol solubility was too low. Recall that in example 1 drospirenone was completely dissolved. Again, the process was conducted in accordance with example 1, including the step of “Granulation—Drying” where a planetary-type mixer was used and the mixture kneaded. The name of the final batch granulated: LPDIMet-16 ml.

In an extra experiment related to this Example 2, in the step “Dissolution of the active ingredients and binder” methanol was added until complete dissolution. The required amount was about 154.3 ml methanol. Such a high necessary amount of methanol may be generally excessive for a relevant industrial granulation process. The process here again was conducted in accordance with example 1, where however in the step “Granulation—Drying” a fluid bed dryer was used and sprayed. The name of the final batch granulated: LPDIMet-154 ml.

### Example 3

### Differential Scanning Calorimetry (DSC)

DSC—using a Shimadzu DSC-60 at about 10° C./min, with a flow of nitrogen at about 30 ml/min as the purge gas—demonstrates the absence of the endothermic signal of fusion characteristic of the crystalline state of the drospirenone in the adsorbate obtained in accordance with Example 1.

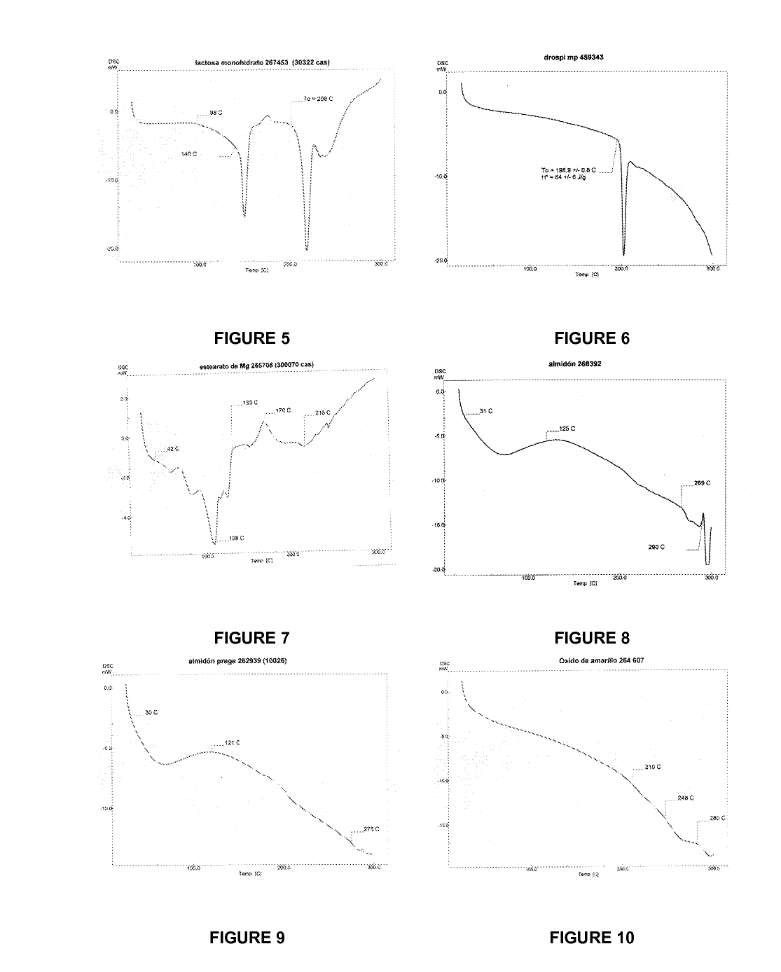

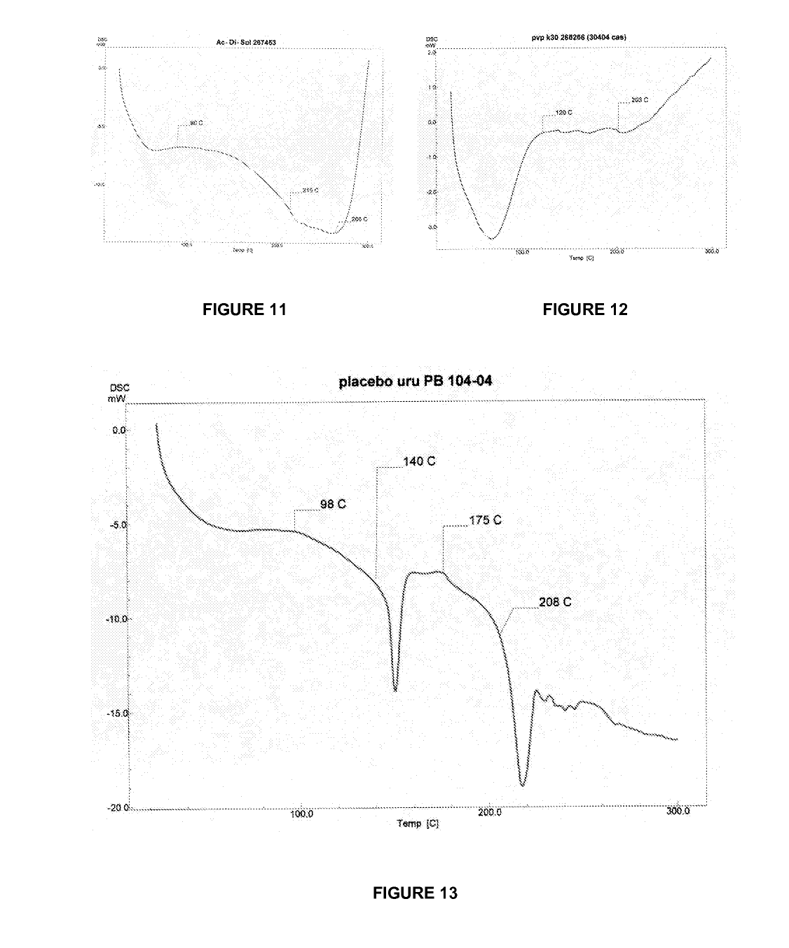

The different inert components and drospirenone were analyzed by DSC before preparing the adsorbate of drospirenone and ethynylestradiol, in accordance with Example 1. FIG. 5 shows a graph of the DSC of lactose monohydrate alone; FIG. 6 shows a graph of the DSC of drospirenone in the crystalline state (temperature of the beginning of the endotherm To=about 198.9 about 0.8° C.). FIG. 7 shows a graph of the DSC of magnesium stearate; FIG. 8 shows a graph of the DSC of corn starch; FIG. 9 shows a graph of the DSC of pregelatinized starch; FIG. 10 shows a graph of the DSC of yellow iron oxide; FIG. 11 shows a graph of the DSC of sodic croscarmellose (AC-Di-Sol); FIG. 12 shows a graph of the DSC of Povidone K30.

FIG. 13 shows a graph of the DSC of the mixture of inert excipient components or placebo with the absence of drospirenone and ethynylestradiol.

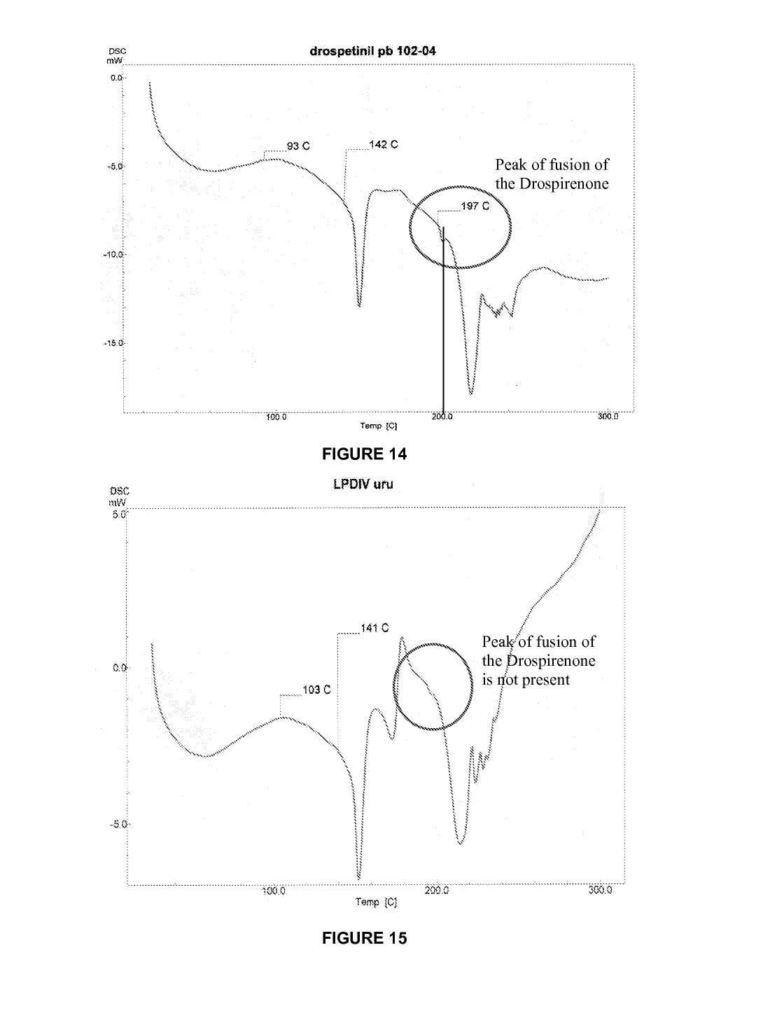

FIG. 14 shows a graph of the DSC of the mixture of the components of the adsorbate in the state of simple mixture, where the signal of fusion of drospirenone in the crystalline state (endotherm of fusion at about 197° C.) has been particularly marked.

FIG. 15 shows a graph of the DSC of the granulate obtained in Example 1 which contains drospirenone and ethynylestradiol adsorbed on inert excipients where one notes the lack of the peak of fusion of the drospirenone in the crystalline state. The lack of the peak indicates the loss of the crystalline state of drospirenone as a result of the process applied in Example 1, where by subjecting the granulate to drying, the presence of the polyvinylpyrrolidone in the drospirenone and ethynylestradiol solution acts through the formation of a tridimensional polymer structure, preventing the crystalline growth of both active compounds, so that the drugs are deposited on the excipient particles in an amorphous state.

### Example 4

### Comparative Dissolution Profiles of Drospirenone

Drospirenone dissolution profiles were obtained from compressed tablets obtained according to Example 1 and “methanol only” comparative example 2B using a dissolution apparatus designated in the U.S. Pharmacopoeia, Edition number 27, Apparatus 2, at 50 rpm and 900 ml of distilled water at 37° C. Similarly, the dissolution profiles of drospirenone were obtained from tablets containing drospirenone in a crystalline state with different sized particles. The results of the amount of drug dissolved at different times are summarized in the following table:

TABLE 2
% of drospirenone dissolved

| | Time (minutes) | | | | | |
|---|---|---|---|---|---|---|
| | 0 | 10 | 20 | 30 | 45 | 60 |
| LP01 | 0 | 31.2% | 48.0% | 59.9% | 69.9% | 77.8% |
| LP02 | 0 | 26.7% | 48.6% | 61.0% | 72.7% | 71.7% |
| LP03 | 0 | 32.6% | 56.2% | 70.7% | 74.3% | 80.5% |
| LPDIVuru | 0 | 80.5% | 97.0% | 101.0% | 102.0% | 102.0% |
| LPDISpray | 0 | 92% | 96% | 95% | 95% | 95% |
| LPDIMet- | 0 | 22% | 29% | 33% | 38% | 42% |
| 16 ml | | | | | | |
| LPDIMet- | 0 | 56% | 73% | 80% | 86% | 88% |
| 154 ml | | | | | | |

### Samples:

LP01: Batch of tablets with non-micronized, crystalline drospirenone with an average particle size of about >100 μm
LP02: Batch of tablets with non-micronized, crystalline drospirenone with an average particle size=about 100 μm
LP03: Batch of tablets with non-micronized, crystalline drospirenone with an average particle size=about 70 μm

LPDIVuru and LPDISpray: Batch of tablets with adsorbates of drospirenone and ethynylestradiol in amorphous form obtained according to Example 1 (i.e. use of mixture of methylene chloride and methanol). For LPDIVuru the granulation was done in a planetary-type mixer and kneaded and for LPDISpray the granulation was done in a fluid bed dryer and sprayed.

LPDIMet-16 ml and LPDIMet-154 ml: Batch of tablets with adsorbates of drospirenone and ethynylestradiol in amorphous form obtained according to comparative Example 2B (i.e. use methanol alone). The first is where 16 ml methanol was used (same amount as in example 1. Drospirenone was not completely dissolved. See example 2B) and the latter is where about 154 ml methanol was used in order to completely dissolve drospirenone.

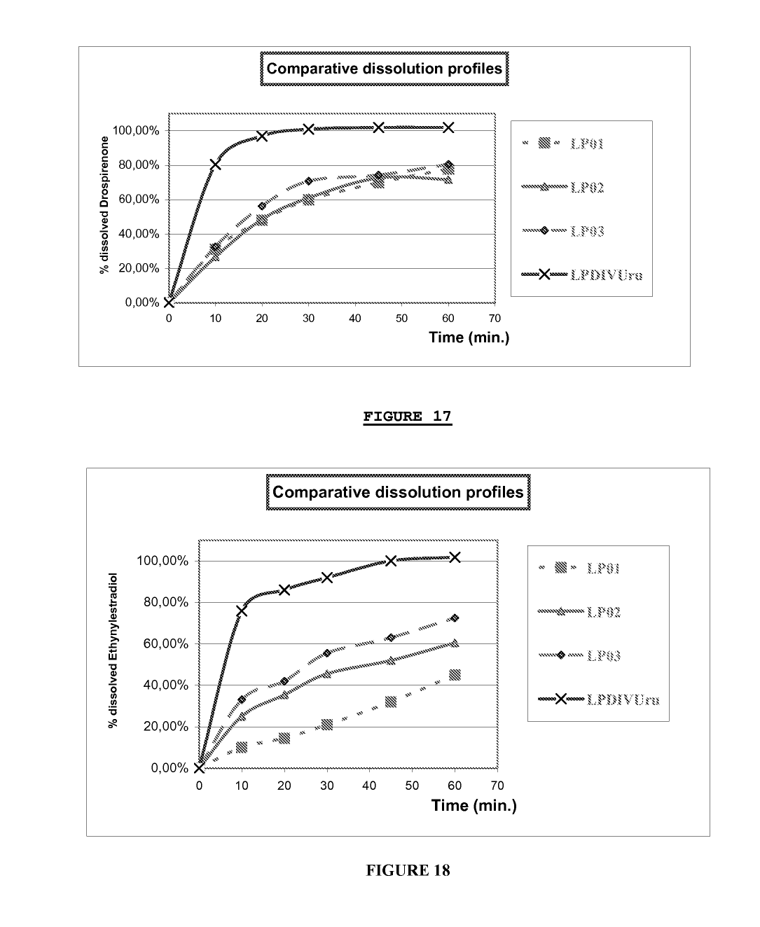

For a better interpretation of the results shown in Table 2, the results are also shown in FIG. 17. From this figure, one can observe that the batches of tablets of drospirenone in crystalline state, with different sizes of particles, show a very slow dissolution rate. However the LPDIVuru batch, in accordance with the present invention, shows a dissolution rate for drospirenone of at least about 80% within the about 20 minutes.

Further, from Table 2 it can be seen that use of methylene chloride as solvent may give significantly better results compared to use of only methanol as solvent.

### Example 5

### Comparative Dissolution Profiles of Ethynylestradiol

Ethynylestradiol dissolution profiles were obtained from compressed tablets obtained according to Example 1 and “methanol only” comparative example 2B using a dissolution apparatus designated in the U.S. Pharmacopoeia, Edition number 27, Apparatus 2, at 50 rpm and 900 ml of distilled water at 37° C. Similarly, the dissolution profiles of ethynylestradiol were obtained from tablets containing ethynylestradiol in a crystalline state with different sizes of particles. The results of the amount of drug dissolved at different times are summarized in the following table:

TABLE 3
% of ethynylestradiol dissolved

| | Time (minutes) | | | | | |
|---|---|---|---|---|---|---|
| | 0 | 10 | 20 | 30 | 45 | 60 |
| LP01 | 0 | 10.0% | 14.5% | 21.0% | 32.0% | 45.0% |
| LP02 | 0 | 25.0% | 35.4% | 45.6% | 52.0% | 60.5% |
| LP03 | 0 | 33.1% | 42.0% | 55.5% | 63.0% | 72.5% |
| LPDIVuru | 0 | 75.9% | 86.0% | 92.0% | 100.1% | 101.8% |
| LPDISpray | 0 | 85% | 89.5% | 92.3% | 95.3% | 102.3% |
| LPDIMet- | 0 | 70% | 75.3% | 81% | 95% | 99% |
| 16 ml | | | | | | |
| LPDIMet- | 0 | 65% | 72.3% | 80.5% | 90.1% | 100.3% |
| 154 ml | | | | | | |

### Samples:

LP01: Batch of tablets with non-micronized, crystalline ethynylestradiol with an average particle size of about >100 μm
LP02: Batch of tablets with non-micronized, crystalline ethynylestradiol with an average particle size=about 90 μm
LP03: Batch of tablets with non-micronized, crystalline ethynylestradiol with an average particle size=about 80 μm

LPDIVuru and LPDISpray: Batch of tablets with adsorbates of drospirenone and ethynylestradiol in amorphous form obtained according to Example 1 (i.e. use of mixture of methylene chloride and methanol). For LPDIVuru the granulation was done in a planetary-type mixer and kneaded and for LPDISpray the granulation was done in a fluid bed dryer and sprayed.

LPDIMet-16 ml and LPDIMet-154 ml: Batch of tablets with adsorbates of drospirenone and ethynylestradiol in amorphous form obtained according to comparative Example 2B (i.e. using methanol alone). The first is where about 16 ml methanol was used (same amount as in example 1—drospirenone not completely dissolved) and the latter is where about 154 ml methanol was used in order to completely dissolve drospirenone.

For a better interpretation of the results shown in Table 3, the results are also shown in FIG. 18. From this figure, it can be observed that the batches of tablets of ethynylestradiol in crystalline state, with different sizes of particles, show a very slow dissolution rate. However, the LPDIVuru batch, in accordance with the present invention, shows a dissolution rate for ethynylestradiol of at least about 80% within the about 20 minutes.

Further, from Table 3 it can be seen that use of methylene chloride as solvent may give better results compared to use of only methanol as solvent.

### Example 6

Due to its low aqueous solubility, drospirenone and ethynylestradiol often show low dissolution rates. The enhancement of rate of dissolution of poorly water-soluble drugs remains one of the most challenging aspects of drug product development. The solid dispersion of poorly water-soluble drugs in water-soluble surface-active carriers enhances drug dissolution and bioavailability.

The nature of drospirenone and ethynylestradiol dispersed in the matrix of polyvinylpyrrolidone K-30 was studied.

In this comparative example, example 1 was repeated but made without use of Povidine (polyvinylpyrrolidone K-30) in the dissolution step. Microphotography by electronic microscopy was obtained. An Electronic Microscopic property of UNLP (Universidad Nacional de La Plata-Argentina) was used in order to obtain the microphotographs.

The microphotograph images of drospirenone and ethynylestradiol crystals obtained by the same manner as in Example I but without polyvinylpyrrolidone K-30 showed that there were micro-crystals precipitating on the excipients. On the contrary, in the microphotograph of granules with drospirenone and ethynylestradiol obtained by wet granulation as in Example 1 (with polyvinylpyrrolidone K-30: matrix of solid dispersion), the image did not exhibit micro-crystal precipitate on the excipients.

Further, making the dissolution test with the USP's apparatus, the results indicated that the drospirenone and ethynylestradiol solid dispersion is released faster from the solid dispersion (with polyvinylpyrrolidone K-30) than from the pure crystalline drug (without polyvinylpyrrolidone K-30).

Accordingly, an improved product may be obtained by adding a water soluble polymer such as polyvinylpyrrolidone K-30 (Povidone) in a manner as described herein.

1. A pharmaceutical composition comprising drospirenone and ethynylestradiol in amorphous form, adsorbed on a therapeutically inert solid support, in combination with pharmaceutically acceptable excipients, wherein the composition contains measurable amounts of the volatile solvent methylene chloride.

2. The pharmaceutical composition in accordance with claim 1, wherein the composition comprises measurable amounts of both of the volatile solvents methylene chloride and methanol.

3. The pharmaceutical composition in accordance with claim 1, wherein the inert solid support is chosen from particles selected from the group consisting of corn starch, pregelatinized starch, lactose, sodic croscarmellose, yellow iron oxide and polyvinylpyrrolidone or mixtures of one or more of these, in combination with pharmaceutically acceptable excipients.

4. The pharmaceutical composition in accordance with claim 1, wherein one or both at least about 80% of drospirenone and at least about 80% of the ethynylestradiol dissolve from said composition within about 20 minutes in a test carried out in a dissolution apparatus according to the U.S. Pharmacopoeia, Edition number 27, Apparatus 2, in 900 ml of distilled water at 37° C. and stirred at 50 rpm.

5. A method for the preparation of the pharmaceutical composition of claim 1, comprising the operations of:
dissolving drospirenone and ethynylestradiol in a volatile solvent or mixture of volatile solvents, wherein at least one volatile solvents, is methylene chloride;
mixing until dissolution;
applying the resulting solution onto a base of solid particles that are therapeutically inert; and
drying the obtained granulated adsorbate.

6. The method of claim 5, wherein the volatile solvent is a mixture of methylene chloride and methanol.

7. The method of claim 6, wherein the mixture of methylene chloride and methanol is one or both of a [9 to 4]:[3 to 0.5] v/v mixture of methylene chloride/methanol and a about a 6:1 v/v mixture of methylene chloride/methanol.

8. (canceled)

9. The method of any of claim 5, wherein a water-soluble polymer is added during either or both between the operations of dissolving and mixing.

10. The method of claim 9, wherein the water-soluble polymer is polyvinylpyrrolidone.

11. The method of claim 5, wherein the base of solid particles is chosen from particles selected from the group consisting of corn starch, pregelatinized starch, lactose, sodic croscarmellose, yellow iron oxide, polyvinylpyrrolidone, or mixtures of one or more of the same.

12. The method of claim 5, wherein the granulated adsorbate is combined with pharmaceutically acceptable excipients for compression into tablet form.

13. A pharmaceutical preparation comprising a number of dose units for daily oral administration for a period of at least about 0.21 consecutive days, wherein each of said dose units comprises from about 1 to about 4 mg of drospirenone and from about 0.01 to about 0.05 mg of ethynylestradiol, wherein said drospirenone and ethynylestradiol are in an amorphous form, adsorbed on the therapeutically inert solid support in combination with pharmaceutically acceptable excipients according to claim 1.

14. The pharmaceutical preparation in accordance with claim 13, comprising drospirenone and ethynylestradiol in amorphous form, adsorbed on a solid support chosen particles selected from the group consisting of corn starch, pregelatinized starch, lactose, sodic croscarmellose, yellow iron oxide, polyvinylpyrrolidone or mixtures of one or more of the same, in combination with pharmaceutically acceptable excipients.

15. The pharmaceutical preparation in accordance with claim 13, wherein at least about 80% of one or both of drospirenone or ethynylestradiol dissolves from said dose units within about 20 minutes, in a test carried out in a dissolution apparatus according to the U.S. Pharmacopoeia, Edition number 27, Apparatus 2, in 900 ml of distilled water at 37° C. and stirred at 50 rpm.

16. (canceled)

17. A method of use of the pharmaceutical composition of claim 1, for the anovulation of one or both of a mammal and a human, wherein the composition comprises an amount of drospirenone corresponding to a daily dosage, of from about 1 mg to about 4 mg, and comprises an amount of ethynylestradiol corresponding to a daily dosage of about 0.01 to about 0.05 mg.

18. The method of claim 17, wherein each of said dose units comprises drospirenone and ethynylestradiol in amorphous form, adsorbed on a solid support chosen from particles selected from the group consisting of corn starch, pregelatinized starch, lactose, sodic croscarmellose, yellow iron oxide, polyvinylpyrrolidone or mixtures of one or more of the same, in combination with pharmaceutically acceptable excipients.

19. (canceled)

20. The method of claim 17, wherein at least about 80% of one or both of drospirenone or ethynylestradiol dissolves from said dose units within about 20 minutes, in a test carried out in a dissolution apparatus according to the U.S. Pharmacopoeia, Edition number 27, Apparatus 2, in 900 ml of distilled water at 37° C. and stirred at 50 rpm.

21. The method of claim 15, wherein the daily oral administration is preferably for a period of at least 21 consecutive days.

22. A pharmaceutical composition of drospirenone and ethynylestradiol in amorphous form, adsorbed on a therapeutically inert solid support, in combination with pharmaceutically acceptable excipients, wherein at least about 80% of one or both of drospirenone or ethynylestradiol dissolve from said composition within about 20 minutes in a test carried out in a dissolution apparatus according to the U.S. Pharmacopoeia, Edition number 27, Apparatus 2, in 900 ml of distilled water at 37° C. and stirred at 50 rpm.

23. A method for the preparation of the pharmaceutical composition of claim 22, comprising the operations of:
i) dissolving drospirenone and ethynylestradiol in a volatile solvent or mixture of volatile solvents,
ii) optionally, adding a water-soluble polymer,
iii) mixing until dissolution,
iv) applying the resulting solution onto a base of solid particles that are therapeutically inert, and
v) drying the obtained granulated adsorbate.

24. The method of claim 23, wherein the volatile solvent or mixture of volatile solvents of step i) is/are solvent(s) that are capable of completely dissolving drospirenone in the following dissolution assay:
16.0 ml of the volatile solvent or mixture of volatile solvents disposed in a stainless steel stirring apparatus, with 0.0306 g of ethynylestradiol (non-micronized with a particle size no less than 50 μm) added under constant stirring, the mixture being shaken/stirred until completely dissolved, with 3.06 g of drospirenone (non-micronized with a particle size no less than 50 μm) added under constant stirring until the drospirenone is completely dissolved.
